(19) United States
(12) Patent Application Publication
(10) Pub. No.: US 2018/0021017 A1
(43) Pub. Date: Jan. 25, 2018

(54) A METHOD AND APPARATUS FOR DISPLAYING A REGION OF INTEREST ON A CURRENT ULTRASONIC IMAGE

(71) Applicant: GENERAL ELECTRIC COMPANY, Schenctady, NY (US)

(72) Inventors: Ye WANG, Wuxi (CN); Jiajiu YANG, Wuxi (CN); Zhixin CHEN, Wuxi (CN); Dongqing CHEN, Wauwatosa, WI (US)

(21) Appl. No.: 15/521,562

(22) PCT Filed: Oct. 24, 2014
(86) PCT No.: PCT/CN2014/089383

§ 371 Date: Aug. 30, 2017

(51) Int. Cl. A61B 8/00

(52) U.S. Cl. CPC A61B 8/463; A61B 8/469; A61B 8/462; A61B 8/0833

(57) ABSTRACT

A method and apparatus for displaying a region of interest in a current ultrasonic image. The method comprises the steps of storing information about a region of interest of a history ultrasonic image corresponding to a current ultrasonic image, reading the information, and displaying a region of interest on the current ultrasonic image according to the information.

### FIELD

The present invention relates to a method and apparatus for processing an ultrasonic image, and particularly to a method and apparatus for displaying a region of interest on a current ultrasonic image.

### BACKGROUND

When performing an ultrasonic examination on a patient, a doctor will usually designate a region of interest (ROI) on an ultrasonic image. Some features capable of reflecting the matters related to nidi are contained within the ROI.

For some symptoms of diseases needing a track and reexamination, the images from two ultrasonic examinations made at different time for the same part may need to be compared. The existing method is simultaneously displaying an ultrasonic image obtained by the current examination and an ultrasonic image obtained by the historic examination stored previously.

However, the existing method cannot display the ROI of the ultrasonic image obtained by the historic examination on the ultrasonic image obtained by the current examination, which is disadvantageous for the doctor to compare the ROIs of the two images.

Therefore, there is a need to provide a method and apparatus for displaying a region of interest on the current ultrasonic image, which is capable of automatically displaying ROI information on the historic ultrasonic image onto the current ultrasonic image.

### SUMMARY

One embodiment of the present invention provides a method for displaying a region of interest on a current ultrasonic image, including: storing information of a region of interest of a historic ultrasonic image corresponding to the current ultrasonic image; reading the information; and displaying the region of interest on the current ultrasonic image according to the information.

Another embodiment of the present invention provides an apparatus for displaying a region of interest on a current ultrasonic image, including: a storing module for storing information of a region of interest of a historic ultrasonic image corresponding to the current ultrasonic image; a reading module for reading the information; and a displaying module for displaying the region of interest on the current ultrasonic image according to the information.

### BRIEF DESCRIPTION OF THE DRAWINGS

The present invention can be understood better in light of the description of embodiments of the present invention with reference to the accompanying drawings, in which:

FIG. 1 illustrates a flow schematic diagram of one embodiment of a method for displaying a region of interest on the current ultrasonic image according to the present invention;

FIG. 2 illustrates a schematic diagram of one embodiment of the format of data stored in information of a region of interest of the historic ultrasonic image in the method of the present invention;

FIG. 3 illustrates a flow schematic diagram of one embodiment of the step of displaying the region of interest on the current ultrasonic image according to the information of the region of interest of the historic ultrasonic image in the method of the present invention;

FIG. 4 illustrates a schematic block diagram of one embodiment of an apparatus for displaying a region of interest on the current ultrasonic image of the present invention;

FIG. 5 illustrates a diagram of a technical effect of contrastively displaying the ROI of the historic ultrasonic image and the ROI of the current ultrasonic image.

### DETAILED DESCRIPTION

Hereafter, a detailed description will be given for preferred embodiments of the present invention. It should be pointed out that in the detailed description of the embodiments, for simplicity and conciseness, it is impossible for the Description to describe all the features of the practical embodiments in details. It should be understood that in the process of a practical implementation of any embodiment, just as in the process of an engineering project or a designing project, in order to achieve a specific goal of the developer and in order to satisfy some system-related or business-related constraints, a variety of decisions will usually be made, which will also be varied from one embodiment to another. In addition, it can also be understood that although the effort made in such developing process may be complex and time-consuming, some variations such as design, manufacture and production on the basis of the technical contents disclosed in the disclosure are just customary technical apparatus in the art for those of ordinary skilled in the art relating to the contents disclosed in the present invention, which should not be regarded as insufficient disclosure of the present invention.

Unless defined otherwise, all the technical or scientific terms used in the Claims and the Description should have the same meanings as commonly understood by one of ordinary skilled in the art to which the present invention belongs. The terms “first”, “second” and the like in the Description and the Claims of the present application for invention do not mean any sequential order, number or importance, but are only used for distinguishing different components. The terms “a”, “an” and the like do not denote a limitation of quantity, but denote the existence of at least one. The terms “comprises”, “comprising”, “includes”, “including” and the like mean that the element or object in front of the “comprises”, “comprising”, “includes” and “including” encompasses the elements or objects and their equivalents illustrated following the “comprises”, “comprising”, “includes” and “including”, but do not exclude other elements or objects. The term “coupled”, “connected” or the like is not limited to being connected physically or mechanically, nor limited to being connected directly or indirectly.

In order to make the purpose, the technical solutions and the advantages of the invention more apparent, the technical solutions of the present invention will be set forth clearly and fully hereinafter in combination with specific embodiments of the invention and the corresponding accompanying drawings. Obviously, the described embodiments are merely part—not all—of the embodiments in the present invention. In view of the embodiments in the present invention, other embodiments made by one of ordinary skilled in the art without inventive work all fall within the scope of protection of the present invention.

When a doctor compares an ultrasonic image obtained by the current examination with an ultrasonic image obtained by the historic examination for the same part, he will usually be concerned about information such as a position of an ROI on the historic ultrasonic image, etc. Embodiments of the present invention will describe a method and apparatus for automatically displaying an ROI on the historic ultrasonic image onto the current ultrasonic image.

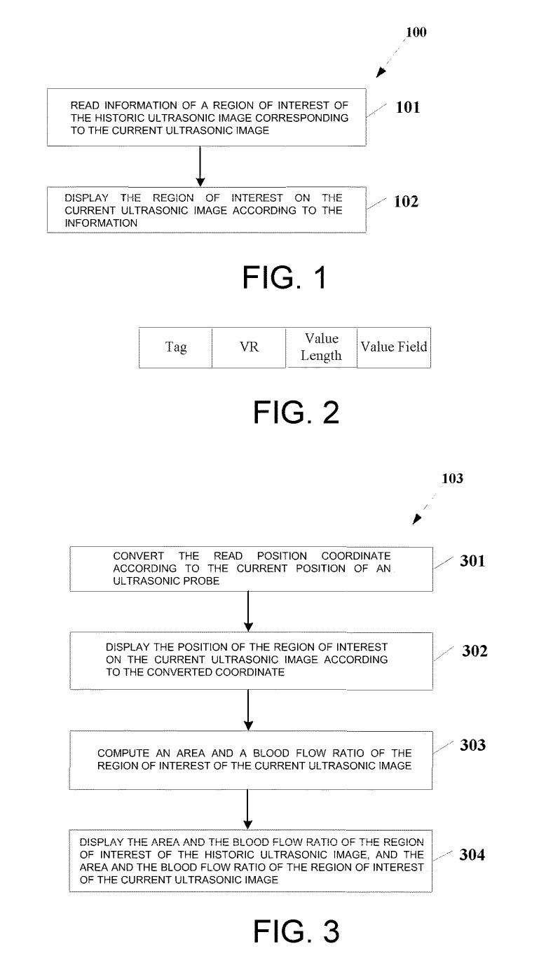

Refer to FIG. 1, which illustrates a flow schematic diagram of one embodiment of a method 100 for displaying a region of interest on the current ultrasonic image of the present invention. The method 100 includes the following steps 101-103.

In step 101, information of a region of interest of the historic ultrasonic image corresponding to the current ultrasonic image is stored.

In one embodiment of the present invention, when the last ultrasonic examination is performed to obtain the historic ultrasonic image, information of a region of interest may be stored in the format as shown in FIG. 2 after the region of interest is determined. Specifically, Tag in FIG. 2 may be used to set a tag for the information of the region of interest. For example, the value of Tag may represent that the information is the exclusive information specific to a certain patient. VR in FIG. 2 may be used to store a type and format of the information of the region of interest. Value Length in FIG. 2 may be used to record the length of the information of the region of interest. Value in FIG. 2 may be used to record the specific content of the information of the region of interest.

In one embodiment of the present invention, the above information of the region of interest may include a position coordinate of the region of interest of the historic ultrasonic image, which may contain coordinate information recording some critical position points of the region of interest. In one embodiment of the present invention, the above information of the region of interest may also include an area of the region of interest of the historic ultrasonic image and a blood flow ratio of the region of interest of the historic ultrasonic image.

In one embodiment of the present invention, the region of interest may be in shape of a rectangle, an ellipse, a closed curve formed by fitting multiple points selected by a user with a curve, or any shape freely drawn by the user.

In step 102, the information is read.

When a re-examination is performed on the same part of the patient with an ultrasonic machine, the ultrasonic image obtained by the historic examination and the current ultrasonic image obtained by the present examination may be simultaneously displayed on a screen for comparison. In one embodiment of the present invention, when a request for displaying the region of interest is received, the information of the region of interest of the historic ultrasonic image stored in step 101 may be read out. Specifically, an ultrasonic operator may first freeze a real-time image obtained by the present ultrasonic examination to obtain a static ultrasonic image, then turn on the comparison and measurement functions, thereby reading the information of the region of interest of the historic ultrasonic image.

In step 103, the region of interest is displayed on the current ultrasonic image according to the information.

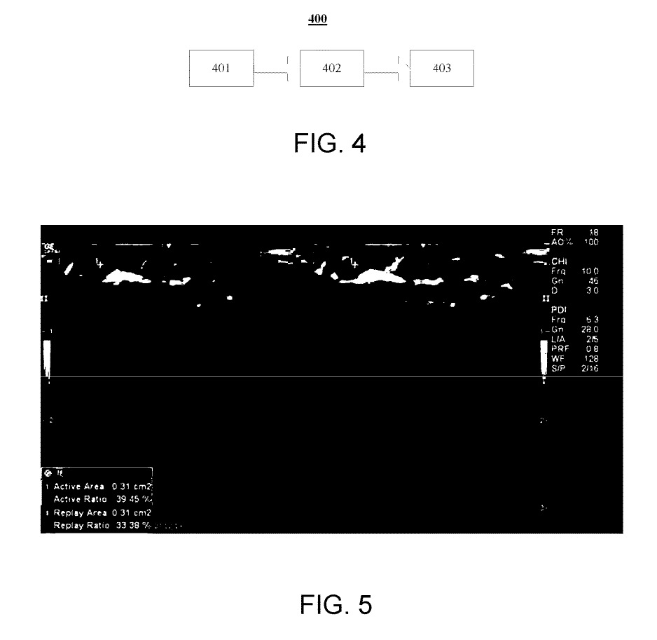

With reference to FIG. 3, in one embodiment of the present invention, step 103 may further include the following sub-steps 301-304.

In sub-step 301, the read position coordinate is converted according to the current position of an ultrasonic probe.

In one embodiment of the present invention, the position coordinate of the region of interest may be contained in the information of the region of interest of the historic ultrasonic image read in step 102. For the position of the ultrasonic probe at the time of generating the current ultrasonic image, the position coordinate may be converted into a coordinate based upon a central position of the current ultrasonic probe.

In sub-step 302, a position of the region of interest is displayed on the current ultrasonic image according to the converted coordinate.

The position coordinate converted in sub-step 301 may be used to display the position of the region of interest on the current ultrasonic image. Certainly, the region of interest may also be displayed on the historic ultrasonic image according to the position coordinate before being converted. In this way, not only two ultrasonic images can be compared with each other, but also the regions of interest on the two ultrasonic images can be compared prominently with each other.

In sub-step 303, an area and a blood flow ratio of the region of interest of the current ultrasonic image are computed.

When the area of the region of interest is computed, for the region of interest having a regular shape, the area thereof may be computed directly with a formula for the area of the shape, while for the region of interest having an irregular shape, the inside thereof may be divided into multiple quadrangles and the area of the region of interest may be computed approximately with the sum of the areas of such quadrangles.

When the blood flow ratio within the region of interest is computed, the pixel points with a difference of RGB three-channel pixel values of the pixel points within the region of interest exceeding a preset threshold may be regarded as the blood flow, thus obtaining a ratio of the blood flow region to the total area of the region of interest.

In sub-step 304, the area and the blood flow ratio of the region of interest of the historic ultrasonic image, and the area and the blood flow ratio of the region of interest of the current ultrasonic image are displayed.

In order to intuitively compare the areas and the blood flow ratios of the regions of interest on the two images, as shown in FIG. 5, in addition to comparatively displaying the two ultrasonic images and the regions of interest (the regions within the dotted lines) thereon on the screen, the area and the blood flow ratio of the region of interest of the historic ultrasonic image and the area and the blood flow ratio of the region of interest of the current ultrasonic image may also be displayed comparatively on the screen. Active Area in the lower-left corner of the screen as shown in FIG. 5 is the area of the region of interest on the current ultrasonic image, Active Ratio is the blood flow ratio of the region of interest on the current ultrasonic image, Replay Area is the area of the region of interest on the historic ultrasonic image, and Replay Ratio is the blood flow ratio of the region of interest on the historic ultrasonic image.

In one embodiment of the present invention, a correlative value of the region of interest of the historic ultrasonic image with the region of interest of the current ultrasonic image may also be computed, so as to inform the user of the size of similarity of the images within the two regions of interest, thereby evaluating the extent of change for the part on which the two ultrasonic examinations are performed. In other words, when the correlative value is very large, it means that the image contents within the two regions of interest are very similar to each other, i.e., the part being examined has not changed a lot. Otherwise, when the correlative value is relatively smaller, it means that the image contents within the two regions of interest are not quite identical, i.e., the part being examined has changed significantly.

So far, the method for displaying the region of interest on the current ultrasonic image according to the embodiment of the present invention has been described. According to the method of the present invention, the information such as the position of the region of interest, the measurement value, etc. on the historic ultrasonic image for comparison can automatically be displayed onto the current ultrasonic image, and a similarity of these two regions of interest can also be computed. This enables the doctor to conveniently and precisely compare the regions of interest on which two examinations are performed.

Similar to the method, the present invention also provides a corresponding apparatus.

FIG. 4 illustrates a schematic block diagram of one embodiment of an apparatus for displaying a region of interest on the current ultrasonic image of the present invention.

As show in FIG. 4, the apparatus 400 may include: a storing module 401 for storing information of a region of interest of a historic ultrasonic image corresponding to the current ultrasonic image; a reading module 402 for reading the information; and a displaying module 403 for displaying the region of interest on the current ultrasonic image according to the information.

In one embodiment of the present invention, the storing module 401 may further include: a tag-setting module for setting a tag for the information; a type-setting module for setting a type for the information; a length-recording module for recording a length of the information; and a content-recording module for recording a content of the information.

In one embodiment of the present invention, the reading module 402 may further include: a receiving module for receiving a request for displaying the region of interest.

In one embodiment of the present invention, the above information includes a position coordinate of the region of interest of the historic ultrasonic image, an area of the region of interest of the historic ultrasonic image, and a blood flow ratio of the region of interest of the historic ultrasonic image.

In one embodiment of the present invention, the displaying module 403 may further include: a coordinate-converting module for converting the read position coordinate according to the current position of an ultrasonic probe; and a position-of-a-region-of-interest-displaying module for displaying a position of the region of interest on the current ultrasonic image according to the converted coordinate.

In one embodiment of the present invention, the displaying module 403 may further include: an area-and-blood-flow-ratio-computing module for computing an area and a blood flow ratio of the region of interest of the current ultrasonic image; and an area-and-blood-flow-ratio-displaying module for displaying the area and the blood flow ratio of the region of interest of the historic ultrasonic image, and the area and the blood flow ratio of the region of interest of the current ultrasonic image.

In one embodiment of the present invention, the apparatus 400 may also include: a correlative-value-computing module for computing a correlative value of the region of interest of the historic ultrasonic image with the region of interest of the current ultrasonic image.

So far, the apparatus for displaying the region of interest on the current ultrasonic image according to the embodiment of the present invention has been described. Similar to the above method, according to the apparatus of the present invention, the information such as the position of the region of interest and the measurement value, etc. on the historic ultrasonic image for comparison can automatically be displayed onto the current ultrasonic image, and a similarity of these two regions of interest can also be computed. This enables the doctor to conveniently and precisely compare the regions of interest on which two examinations are performed.

The above descriptions are merely embodiments of the present invention and are not intended to restrict the scope of the present invention. All kinds of variations and modifications could be made to the present invention to those skilled in the art. Any modifications, alternatives, and improvements made within the spirit and the principles of the present invention shall fall within the scope of the appended claims.

1. A method for displaying a region of interest on a current ultrasonic image, comprising:
storing information about a region of interest in a historic ultrasonic image corresponding to the current ultrasonic image;
reading the information about the region of interest; and
displaying the region of interest on the current ultrasonic image according to the information.

2. The method according to claim 1, wherein the step of storing information about a region of interest further comprises:
setting a tag for the information;
setting a type for the information;
recording a length of the information; and
recording a content of the information.

3. The method according to claim 1, wherein the information about a region of interest comprises a position coordinate of the region of interest in the historic ultrasonic image, an area of the region of interest of the historic ultrasonic image, and a blood flow ratio of the region of interest of the historic ultrasonic image.

4. The method according to claim 1, wherein the step of reading the information about a region of interest further comprises receiving a request for displaying the region of interest.

5. The method according to claim 3, wherein the step of displaying the region of interest further comprises:
converting the read position coordinate according to a current position of an ultrasonic probe; and
displaying a position of the region of interest on said current ultrasonic image according to the converted coordinate.

6. The method according to claim 5, wherein said step of displaying the region of interest further comprises:
computing an area and a blood flow ratio of the region of interest of the current ultrasonic image; and
displaying the area and the blood flow ratio of the region of interest of the historic ultrasonic image, and the area and the blood flow ratio of the region of interest of the current ultrasonic image.

7. The method according to claim 1, further comprising computing a correlative value of the region of interest of the historic ultrasonic image with the region of interest of the current ultrasonic image.

8. An apparatus for displaying a region of interest on a current ultrasonic image, comprising:
a storing module configured to store information of a region of interest in a historic ultrasonic image corresponding to the current ultrasonic image;
a reading module configured to read the information; and
a displaying module configured to display the region of interest on the current ultrasonic image according to the information.

9. The apparatus according to claim 8, wherein the storing module further comprises:
a tag-setting module for setting a tag for said information;
a type-setting module for setting a type for said information;
a length-recording module for recording a length of said information; and
a content-recording module for recording a content of said information.

10. The apparatus according to claim 8, wherein said reading module further comprises:
a receiving module for receiving a request for displaying the region of interest.

11. The apparatus according to claim 8, wherein said information comprises a position coordinate of the region of interest of the historic ultrasonic image, an area of the region of interest of the historic ultrasonic image, and a blood flow ratio of the region of interest of the historic ultrasonic image.

12. The apparatus according to claim 11, wherein said displaying module further comprises:
a coordinate-converting module for converting the read position coordinate according to a current position of an ultrasonic probe; and
a position-of-a-region-of-interest-displaying module for displaying a position of the region of interest on said current ultrasonic image according to the converted coordinate.

13. The apparatus according to claim 11, wherein said displaying module further comprises:
an area-and-blood-flow-ratio-computing module for computing an area and a blood flow ratio of the region of interest of the current ultrasonic image; and
an area-and-blood-flow-ratio-displaying module for displaying the area and the blood flow ratio of the region of interest of the historic ultrasonic image, and the area and the blood flow ratio of the region of interest of the current ultrasonic image.

14. The apparatus according to claim 8, further comprising:
a correlative-value-computing module for computing a correlative value of the region of interest of the historic ultrasonic image with the region of interest of the current ultrasonic image.
